(19) United States
(12) Patent Application Publication
(10) Pub. No.: US 2003/0108576 A1
(43) Pub. Date: Jun. 12, 2003

(54) Composition

(75) Inventor: Cristina Bielli, Hillsborough, NJ (US)

Correspondence Address: Colgate-Palmolive Company, 909 River Road, P.O. Box 1343, Piscataway, NJ, 08855-1343, US

(73) Assignee: Colgate-Palmolive Company

(21) Appl. No.: 10/026,274

(22) Filed: Oct. 25, 2001

(51) Int. Cl. A61K 7/06; A61K 7/00; A61K 31/355

(52) U.S. Cl. 424/401; 514/458; 424/70.1

(57) ABSTRACT

An aqueous liquid composition which comprises:

a. a surfactant or mixture of surfactants in quantities effective to cleanse skin, and

b. an amount of Vitamin E in quantities sufficient to protect skin, and

c. a Vitamin E stabilizing effective amount of 2-tertiary butyl hydroquinone, and

d. the balance water.

### BACKGROUND OF THE INVENTION

Vitamin E itself provides immediate benefit to human skin, for example protection from the deleterious effects of sun, air pollutants such as ozone, and the like. However, this form of the vitamin is very unstable in aqueous compositions and is generally not used in personal care products. Various derivatives of the vitamin are more stable, particularly the esters such as Vitamin E acetate. However, these derivatives are not active until the enzymes found naturally in the skin convert the derivative, particularly esters such as Vitamin E acetate, into active Vitamin E.

An antioxidant has now been discovered which brings about significantly more stabilization of Vitamin E than brought about by the usage of other known antioxidants.

### SUMMARY OF THE INVENTION

In accordance with the invention, there is an aqueous liquid composition, which comprises:

a. a surfactant or mixture of surfactants in quantities effective to cleanse skin, and

b. an amount of Vitamin E in quantities sufficient to protect skin, and

c. a Vitamin E stabilizing effective amount of 2-tertiary butyl hydroquinone, and

d. the balance water.

A further aspect of the invention is an aqueous liquid composition, which comprises:

a. at least about 1 wt. % of a skin cleansing surfactant or a mixture of surfactants,

b. at least about 0.01 wt. % of the composition of Vitamin E, and

c. at least about 0.01 wt. % of 2-tertiary butyl hydroquinone, and

d. at least about 70 wt. % water

### DETAILED DESCRIPTION OF THE INVENTION

An aqueous liquid composition includes gels as well as liquids. The general overall meaning is a non-solid. Viscosities of about 100 to about 60,000 centipoise, measured at room temperature using a Brookfield viscometer with a No. 5 spindle at 20 rpm are intended to be covered as well as gels which, for example, are squeezed from a tube.

Any surfactant or mixture thereof which can cleanse skin at an appropriate concentration can be employed.

The surfactant can be anionic, zwitterionic, nonionic, amphoteric, or cationic, preferably anionic. Soap, a long chain alkyl or alkenyl, branched or normal carboxylic acid salt, the salt being formed from a cation such as sodium, potassium, ammonium or substituted ammonium can be present in the composition as an example of an anionic surfactant. Exemplary of long chain alkyl or alkenyl are from about 8 to about 22 carbon atoms in length, specifically about 10 to about 20 carbon atoms in length, more specifically alkyl and most specifically normal, or normal with little branching. Small quantities of olefinic bond(s) may be present in the predominantly alkyl sections, particularly if the source of the “alkyl” group is obtained from a natural product such as tallow, coconut oil and the like. Because of its potential harshness soap need not be a preferred surfactant and can be omitted from the composition.

Other surfactants can be present in the composition as well. Examples of other anionic surfactants include but are not limited to alkyl sulfates, anionic acyl sarcosinates, methyl acyl taurates, N-acyl glutamates, acyl isethionates, alkyl sulfosuccinates, alkyl phosphate esters, ethoxylated alkyl phosphate esters, trideceth sulfates, protein condensates, mixtures of ethoxylated alkyl sulfates and the like.

Alkyl chains for these surfactants are C₈-C₂₂, preferably C₁₀-C₁₈, more preferably C₁₂-C₁₄.

Anionic non-soap surfactants can be exemplified by the alkali metal salts of organic sulfate having in their molecular structure an alkyl radical containing from about 8 to about 22 carbon atoms and a sulfonic acid or sulfuric acid ester radical (included in the term alkyl is the alkyl portion of higher acyl radicals). Preferred are the sodium, ammonium, potassium or triethanolamine alkyl sulfates, especially those obtained by sulfating the higher alcohols (C₈-C₁₈ carbon atoms), sodium coconut oil fatty acid monoglyceride sulfates and sulfonates; sodium or potassium salts of sulfuric acid esters of the reaction product of 1 mole of a higher fatty alcohol (e.g., tallow or coconut oil alcohols) and 1 to 12 moles of ethylene oxide; sodium or potassium salts of alkyl phenol ethylene oxide ether sulfate with 1 to 10 units of ethylene oxide per molecule and in which the alkyl radicals contain from 8 to 12 carbon atoms, sodium alkyl glyceryl ether sulfonates; the reaction product of fatty acids having from 10 to 22 carbon atoms esterified with isethionic acid and neutralized with sodium hydroxide; water soluble salts of condensation products of fatty acids with sarcosine; and others known in the art.

Zwitterionic surfactants can be exemplified by those which can be broadly described as derivatives of aliphatic quaternary ammonium, phosphonium, and sulfonium compounds, in which the aliphatic radicals can be straight chain or branched and wherein one of the aliphatic substituents contains from about 8 to 18 carbon atoms and one contains an anionic water-solubilizing group, e.g., carboxy, sulfonate, sulfate, phosphate, or phosphonate. A general formula for these compounds is:

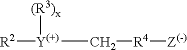

wherein R² contains an alkyl, alkenyl, or hydroxy alkyl radical of from about 8 to about 18 carbon atoms, from 0 to about 10 ethylene oxide moieties and from 0 to 1 glyceryl moiety; Y is selected from the group consisting of nitrogen, phosphorus, and sulfur atoms; R3 is an alkyl or monohydroxyalkyl group containing 1 to about 3 carbon atoms; X is 1 when Y is a sulfur atom and 2 when Y is a nitrogen or phosphorus atom, R⁴ is an alkylene or hydroxyalkylene of from 0 to about 4 carbon atoms and Z is a radical selected from the group consisting of carboxylate, sulfonate, sulfate, phosphonate, and phosphate groups.

Examples include: 4-[N,N-di(2-hydroxyethyl)-N-octadecylammonio]-butane-1-carboxylate; 5-[S-3-hydroxypropyl-S-hexadecylsulfonio]-3 hydroxypentane-1-sulfate; 3-[P,P-P-diethyl-P 3,6,9 trioxatetradecyl-phosphonio]-2-hydroxypropane-1-phosphate; 3-[N,N-dipropyl-N-3 dodecoxy-2-hydroxypropylammonio]-propane-1-phosphonate; 3-(N,N-di-methyl-N-hexadecylammonio) propane-1-sulfonate; 3-(N,N-dimethyl-N-hexadecylammonio)-2-hydroxypropane-1-sulfonate; 4-(N,N-di(2-hydroxyethyl)-N-(2 hydroxydodecyl) ammonio]-butane-1-carboxylate; 3-[S-ethyl-S-(3-dodecoxy-2-hydroxypropyl)sulfonio]-propane-1-phosphate; 3-(P,P-dimethyl-P-dodecylphosphonio)-propane-1-phosphonate; and 5-[N,N-di(3-hydroxypropyl)-N-hexadecylammonio]-2-hydroxy-pentane-1-sulfate.

Examples of amphoteric surfactants which can be used in the compositions of the present invention are those which can be broadly described as derivatives of aliphatic secondary and tertiary amines in which the aliphatic radical can be straight chain or branched and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecylaminopropionate, sodium 3-dodecylaminopropane sulfonate, N-alkyltaurines, such as the one prepared by reacting dodecylamine with sodium isethionate according to the teaching of U.S. Pat. No. 2,658,072, N-higher alkyl aspartic acids, such as those produced according to the teaching of U.S. Pat. No. 2,438,091, and the products sold under the trade name “Miranol” and described in U.S. Pat. No. 2,528,378. Other amphoterics such as betaines are also useful in the present composition.

Examples of betaines useful herein include the high alkyl betaines such as coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl)carboxy methyl betaine, stearyl bis-(2-hydroxypropyl) carboxymethyl betaine, oleyl dimethyl gamma-carboxypropyl betaine, lauryl bis-(2-hydro-xypropyl) alpha-carboxyethyl betaine, etc. The sulfobetaines may be represented by coco dimethyl sulfopropyl betaine, stearyl dimethyl sulfopropyl betaine, amido betaines, amidosulfobetaines, and the like.

Many cationic surfactants are known to the art. By way of example, the following may be mentioned:

stearyldimenthylbenzyl ammonium chloride;

dodecyltrimethylammonium chloride;

nonylbenzylethyldimethyl ammonium nitrate;

tetradecylpyridinium bromide;

laurylpyridinium chloride;

cetylpyridinium chloride

laurylpyridinium chloride;

laurylisoquinolium bromide;

ditallow(Hydrogenated)dimethyl ammonium chloride;

dilauryldimethyl ammonium chloride; and stearalkonium chloride.

Additional cationic surfactants are disclosed in U.S. Pat. No. 4,303,543 see column 4, lines 58 and column 5, lines 1-42, incorporated herein by references. Also see CTFA Cosmetic Ingredient Dictionary, 4th Edition 1991, pages 509-514 for various long chain alkyl cationic surfactants; incorporated herein by references.

Nonionic surfactants can be broadly defined as compounds produced by the condensation of alkylene oxide groups (hydrophilic in nature) with an organic hydrophobic compound, which may be aliphatic or alkyl aromatic in nature. Examples of preferred classes of nonionic surfactants are:

1. The polyethylene oxide condensates of alkyl phenols, e.g., the condensation products of alkyl phenols having an alkyl group containing from about 6 to 12 carbon atoms in either a straight chain or branched chain configuration, with ethylene oxide, the said ethylene oxide being present in amounts equal to 10 to 60 moles of ethylene oxide per mole of alkyl phenol. The alkyl substituent in such compounds may be derived from polymerized propylene, diisobutylene, octane, or nonane, for example.

2. Those derived from the condensation of ethylene oxide with the product resulting from the reaction of propylene oxide and ethylene diamine products which may be varied in composition depending upon the balance between the hydrophobic and hydrophilic elements which is desired. For example, compounds containing from about 40% to about 80% polyoxyethylene by weight and having a molecular weight of from about 5,000 to about 11,000 resulting from the reaction of ethylene oxide groups with a hydrophobic base constituted of the reaction product of ethylene diamine and excess propylene oxide, said base having a molecular weight of the order of 2,500 to 3,000, are satisfactory.

3. The condensation product of aliphatic alcohols having from 8 to 18 carbon atoms, in either straight chain or branched chain configuration with ethylene oxide, e.g., a coconut alcohol ethylene oxide condensate having from 10 to 30 moles of ethylene oxide per mole of coconut alcohol, the coconut alcohol fraction having from 10 to 14 carbon atoms. Other ethylene oxide condensation products are ethoxylated fatty acid esters of polyhydric alcohols (e.g., Tween 20-polyoxyethylene (20) sorbitan monolaurate).

4. Long chain tertiary amine oxides corresponding to the following general formula:

R₁R₂R₃N→0

wherein R₁ contains an alkyl, alkenyl or monohydroxy alkyl radical of from about 8 to about 18 carbon atoms, from 0 to about 10 ethylene oxide moieties, and from 0 to 1 glyceryl moiety, and, R₂ and R₃ contain from 1 to about 3 carbon atoms and from 0 to about 1 hydroxy group, e.g., methyl, ethyl, propyl, hydroxy ethyl, or hydroxy propyl radicals. The arrow in the formula is a conventional representation of a semipolar bond. Examples of amine oxides suitable for use in this invention include dimethyldodecylamine oxide, oleyl-di(2-hydroxyethyl) amine oxide, dimethyloctylamine oxide, dimethyldecylamine oxide, dimethyltetradecylamine oxide, 3,6,9 trioxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, dimethylhexadecylamine oxide.

5. Long chain tertiary phosphine oxides corresponding to the following general formula:

RR′R″P→0

wherein R contains an alkyl, alkenyl or monohydroxyalkyl radical ranging from 8 to 20 carbon atoms in chain length, from 0 to about 10 ethylene oxide moieties and from 0 to 1 glyceryl moiety and R′ and R″ are each alkyl or monohydroxyalkyl groups containing from 1 to 3 carbon atoms. The arrow in the formula is a conventional representation of a semipolar bond. Examples of suitable phosphine oxides are: dodecyldimethylphosphine oxide, tetradecylmethylethylphosphine oxide, 3,6,9-trioxaoctadecyldimethylphosphine oxide, cetyldimethylphosphine oxide, 3-dodecoxy-2-hydroxypropyldi(2-hydroxyethyl) phosphine oxide stearyldimethylphosphine oxide, cetylethyl propylphosphine oxide, oleyldiethylphosphine oxide, dodecyldiethylphosphine oxide, tetradecyldiethylphosphine oxide, dodecyldipropylphosphine oxide, dodecyldi(hydroxymethyl)phosphine oxide, dodecyldi(2-hydroxyethyl)phosphine oxide, tetradecylmethyl-2-hydroxypropylphosphine oxide, oleyldimethylphosphine oxide, 2-hydroxydodecyldimethylphosphine oxide.

6. Long chain dialkyl sulfoxides containing one short chain alkyl or hydroxy alkyl radical of 1 to about 3 carbon atoms (usually methyl) and one long hydrophobic chain which contain alkyl, alkenyl, hydroxy alkyl, or keto alkyl radicals containing from about 8 to about 20 carbon atoms, from 0 to about 10 ethylene oxide moieties and from 0 to 1 glyceryl moiety. Examples include: octadecyl methyl sulfoxide, 2-ketotridecyl methyl sulfoxide, 3,6,9-trioxaoctadecyl 2-hydroxyethyl sulfoxide, dodecyl methyl sulfoxide, oleyl 3-hydroxypropyl sulfoxide, tetradecyl methyl sulfoxide, 3 methoxytridecylmethyl sulfoxide, 3-hydroxytridecyl methyl sulfoxide, 3-hydroxy-4-dodecoxybutyl methyl sulfoxide.

7. Alkylated polyglycosides wherein the alkyl group is from about 8 to about 20 carbon atoms, preferably about 10 to about 18 carbon atoms and the degree of polymerization of the glycoside is from about 1 to about 3, preferably about 1.3 to about 2.0.

Vitamin E, sometimes known as tocopherol or alpha tocopherol, is a vitamin well known for its skin protection function. This protection is immediate or almost immediate upon deposition upon skin as opposed to, for example various derivatives such as esters similar to Vitamin E acetate, for example carboxylic acid group, having 1 to 20 carbon atoms, which require cleavage by skin enzymes to produce the active Vitamin E. Although directed primarily to protecting Vitamin E from degradation, the tertiary butyl hydroquinone can also be effective against other desirable vitamins beneficial to the skin such as Vitamin A and Vitamin C, and their various derivatives useful in skin contacting compositions.

2-tertiary butyl hydroquinone is the antioxidant which helps preserve the presence of Vitamin E as well as fostering the continued presence of Vitamins A, C and their beneficial derivatives, if present in the composition.

The quantity of surfactant or mixture thereof is as wt. % of the composition at least about 1, 2, 3, 4, 5, 6, 7 or 8%. The maximum amount of surfactant or mixture thereof is not that significant but is generally no more than about 25, 20, or even 15 wt. % of the composition.

The minimum quantity of Vitamin E present in the composition is at least about 0.01, 0.015, 0.02, 0.03, 0.04 and 0.05 wt. %. The maximum amount is a non-toxic quantity. Generally, no more than about 2, 1, 0.75 or 0.5 wt. % can be employed.

The 2-tertiary butyl hydroquinone is present in quantities of at least about 0.005, 0.01, or 0.02 wt. % of the compositions. Maximum quantities are about 0.1, 0.075, or 0.06 wt. % of the composition.

Other material can be present in the compositions such as colorants, preservatives, fragrances, emollients, thickeners, gellants, antibacterials, and the like. Examples of preservatives include dimethylol dimethyl hydantoin, the Kathons and the like; fragrance, particularly those with aromatic groups, phenols and the like useful for dissolving the tertiary butyl hydroquinone; thickeners and gellants such as cellulosic gums such as carboxymethyl cellulose, hydroxyethyl cellulose, methyl cellulose, and the like; acrylic acid polymers and copolymers; acrylic emulsion polymers; acrylamides, xanthan gum; inorganic clays; polyethylene glycols; and the like. Antimicrobials such as triclosan and triclocarban can be employed as well.

However, exempting sodium chloride chelants such as ethylene diamine tetra acetic acid (EDTA) can be particularly helpful.

Since the 2-tertiary butyl hydroquinone can discolor in the presence of proteins, sodium salts and alkaline pH, these materials are preferably absent in the composition.

The pH of the composition is significant and is generally greater than about 4 and less than about 8, preferably less than about 7. At a pH of about 4, the composition can become opaque as opposed to a preferred minimum pH of about 5 wherein the composition can remain clear. Above about 8, the 2-tertiary butyl hydroquinone loses effectiveness.

The compositions of the invention are readily prepared by dissolving the vitamin(s) and tertiary butyl hydroquinone in a fragrance when employed in the composition. If a fragrance is not employed, the hydroquinones can be well dispersed in the final composition.

The following examples are prepared.

| Component | Wt. % |
|---|---|
| Sodium Laureth Sulfate | 4.0 |
| Cocoamidopropyl Betaine | 1.2 |
| Decylpolyglucoside | 1.5 |
| PEG 80/PEG30 Glyceryl Monococoate | 6.0 |
| DMDM Hydantoin | 0.22 |
| Perfume | 0.4 |
| Citric Acid | 0.065 |
| EDTA | 0.043 |
| Vitamin E | 0.075 |
| Vitamin E Acetate | 0.05 |
| Vitamin A Palmitate | 0.01 |
| Antioxidant* | 0.05 |
| Distilled Water | Balance |

*Example 1—2-tertiary butyl hydroquinone
Example 2—Butylated hydroxy toluene
Example 3—Propyl gallate
Example 4—Tetrakis [methylene(3,5 dl-tert-butyl-4-hydroxycinnamate)] methane
Example 5—0.2 EDTA
Example 6—Control 0.044 EDTA

Samples of each example are put on stability aging using clear PET packaging at room temperature, 77° F.; high temperature, 110° F.; and in the window for natural sunlight exposure. Samples are evaluated for vitamin content initially, then again at 4,8, and 13 weeks. Below are the results.

TABLE 1
Vitamin E Stability Data
Sunlight

| | 0 | 4 | 8 | 13 | % Retained |
|---|---|---|---|---|---|
| TBHQ (Example 1) | 0.069 | 0.047* | 0.063 | 0.059 | 85.5 |
| BHT (Example 2) | 0.07 | 0.054 | 0.043 | 0.027 | 38.6 |
| PG (Example 3) | 0.07 | 0.026 | 0.007 | 0 | 0.0 |
| IRG (Example 4) | 0.077 | 0.051 | 0.044 | 0.024 | 31.2 |
| VERS (Example 5) | 0.071 | 0.049 | 0.032 | 0.012 | 16.9 |
| Control | 0.075 | 0.046 | 0.032 | 0.015 | 20.0 |

TABLE 2
Vitamin E Stability Data
77° F.

| | 0 | 4 | 8 | 13 | % Retained |
|---|---|---|---|---|---|
| TBHQ (Example 1) | 0.069 | 0.051* | 0.066 | 0.064 | 92.8 |
| BHT (Example 2) | 0.07 | 0.068 | 0.068 | 0.071 | 101.4 |
| PG (Example 3) | 0.07 | 0.07 | 0.066 | 0.067 | 95.7 |
| IRG (Example 4) | 0.077 | 0.072* | 0.077 | 0.079 | 102.6 |
| VERS (Example 5) | 0.071 | 0.061* | 0.07 | 0.068 | 95.8 |
| Control | 0.075 | 0.76 | 0.076 | 0.077 | 102.7 |

TABLE 3
Vitamin E Stability Data
110° F.

| | 0 | 4 | 8 | 13 | % Retained |
|---|---|---|---|---|---|
| TBHQ (Example 1) | 0.069 | 0.061 | 0.063 | 0.058 | 84.1 |
| BHT (Example 2) | 0.07 | 0.072 | 0.072 | 0.075 | 107.1 |
| PG (Example 3) | 0.07 | 0.068 | 0.06 | 0.061 | 87.1 |
| IRG (Example 4) | 0.077 | 0.077 | 0.063 | 0.076 | 98.7 |
| VERS (Example 5) | 0.071 | 0.068 | 0.064 | 0.063 | 88.7 |
| Control | 0.075 | 0.051* | 0.068 | 0.074 | 98.7 |

*Appears to be incorrect because of 8 and 13 week numbers, but insufficient sample left to reevaluate.

As shown by the data, the antioxidant of the invention 2-tertiary butyl hydroquinone provides substantially more retention of vitamin E in sunlight than any of the other test antioxidants and is essentially equivalent to the other antioxidants at both 77° F. and 110° F.

Vitamin E acetate stability is also tested in the same manner with all of the antioxidants. Equivalent results are obtained for all the antioxidants, each one showing no loss of Vitamin E acetate.

These tests show the marked differentiation in Vitamin E retention with 2-tertiary butyl hydroquinone as compared to the equivalency of results of all tested antioxidants for Vitamin E acetate.

1. An aqueous liquid composition which comprises:
a. a surfactant or mixture of surfactants in quantities effective to cleanse skin, and
b. an amount of Vitamin E in quantities sufficient to protect skin, and
c. a Vitamin E stabilizing effective amount of 2-tertiary butyl hydroquinone, and
d. the balance water.

2. The composition in accordance with claim 1 wherein at least about 1 wt. % of surfactant is present.

3. The composition in accordance with claim 1 wherein at least about 0.01 wt. % of Vitamin E is present.

4. The composition in accordance with claim 1 wherein about 0.005 wt. % of 2-tertiary butyl hydroquinone is present.

5. The composition in accordance with claim 2 wherein at least about 0.01 wt. % Vitamin E is present.

6. The composition in accordance with claim 2 wherein at least about 0.005 wt. % 2-tertiary butyl hydroquinone is present.

7. The composition in accordance with claim 6 wherein at least about 0.01 wt. % of Vitamin E is present.

8. An aqueous liquid composition which comprises:
a. at least about 1 wt. % of a skin cleansing surfactant or a mixture of surfactants,
b. at least about 0.01 wt. % of the composition of Vitamin E, and
c. at least about 0.01 wt. % of 2-tertiary butyl hydroquinone, and
d. at least about 70 wt. % water.
